(19) United States
(12) Patent Application Publication
(10) Pub. No.: US 2015/0010498 A1
(43) Pub. Date: Jan. 8, 2015

(54) Immunocytokines for Cancer Treatment in Combination with Chemotherapeutic Agents

(71) Applicant: Philogen S.p.A., Siena (IT)

(72) Inventors: Eveline Trachsel, Zurich (CH); Manuela Kaspar, Zurich (CH); Dario Neri, Zurich (CH); Jessica Marlind, Zurich (CH)

(21) Appl. No.: 14/457,522

(22) Filed: Aug. 12, 2014

Related U.S. Application Data
(63) Continuation of application No. 13/679,374, filed on Nov. 16, 2012, now abandoned.
Application No. 13/679,374 is a continuation of application No. 12/452,264, filed on Dec. 22, 2009, now abandoned.
(60) Provisional application No. 60/946,761, filed on Jun. 28, 2007.

(51) Int. Cl. A61K 47/48; A61K 31/337

(52) U.S. Cl. CPC A61K 47/48569; A61K 31/337; A61K 47/48584; A61K 47/48592
USPC 424/85.2

(57) ABSTRACT

This invention relates to the treatment of cancer using anti-cancer agents, such as doxorubicin or paclitaxel, in combination with antibody-interleukin 2 (IL2) conjugates which target tenascin-C.

This invention relates to the treatment of cancer using a combination of chemotherapeutic agents and immunocytokines.

Tenascin-C is a large hexameric glycoprotein of the extracellular matrix which modulates cellular adhesion. It is involved in processes such as cell proliferation and cell migration and is associated with changes in tissue architecture as occurring during morphogenesis and embryogenesis as well as under tumorigenesis or angiogenesis.

A strong over-expression of the large isoform of tenascin-C has been reported for a number of tumors [Borsi 1992 supra], and monoclonal antibodies specific for domains Al and D, respectively, have been extensively characterised in the clinic [Riva P et al. Int J Cancer 1992; 51:7-13, Riva P et al. Cancer Res 1995; 55:5952s-5956s, Paganelli G et al Eur J Nucl Med 1994; 21:314-321, Reardon D A et al. J Clin Oncol 2002; 20:1389-1397, Signer D D et al. J Clin Oncol 1998; 16:2202-2212.

Human monoclonal antibody fragments specific to tenascin-C are described in WO2006/050834 and shown to bind preferentially to tumor tissue relative to normal tissue. These antibodies are useful, for example, in delivering toxins, such as cytokines, specifically to tumour cells.

The present inventors have discovered that antibody-cytokine conjugates which target tenascin-C have an unexpected synergy with anti-cancer compounds such as doxorubicin and paclitaxel in the treatment of cancer.

An aspect of the invention provides a method of treating cancer comprising:
administering an anti-cancer compound and an antibody-interleukin 2 (IL2) conjugate to an individual in need thereof,
wherein the antibody-IL2 conjugate comprises IL2 conjugated to an antibody which specifically binds to tenascin-C.

Other aspects of the invention provide an anti-cancer compound for use in a method of treating cancer comprising administering an anti-cancer compound in combination with an antibody-IL2 conjugate comprising interleukin 2 (IL2) conjugated to an antibody which specifically binds to tenascin-C to an individual in need thereof, and the use of a an anti-cancer compound in the manufacture of a medicament for use in a method of treating cancer comprising administering the anti-cancer compound in combination with an antibody-IL2 conjugate to an individual in need thereof,
said antibody-IL2 conjugate comprising interleukin 2 (IL2) conjugated to an antibody which specifically binds to tenascin-C.

Other aspects of the invention provide an antibody-IL2 conjugate comprising interleukin 2 (IL2) conjugated to an antibody which specifically binds to tenascin-C for use in a method of treating cancer comprising administering the antibody-IL2 conjugate in combination with an anti-cancer compound to an individual in need thereof and the use of an antibody-IL2 conjugate comprising interleukin 2 (IL2) conjugated to an antibody which specifically binds to tenascin-C in the manufacture of a medicament for use in a method of treating cancer comprising administering the antibody-IL2 conjugate in combination with the anti-cancer compound to an individual in need thereof.

Other aspects of the invention provide a combination of an anti-cancer compound and an antibody-IL2 conjugate comprising interleukin 2 (IL2) conjugated to an antibody which specifically binds to tenascin-C for use in a method of treating cancer comprising administering the antibody-IL2 conjugate and the anti-cancer compound to an individual in need thereof and the use of a combination of an anti-cancer compound and an antibody-IL2 conjugate comprising interleukin 2 (IL2) conjugated to an antibody which specifically binds to tenascin-C in the manufacture of a medicament for use in a method of treating cancer comprising administering the antibody-IL2 conjugate and the anti-cancer compound to an individual in need thereof.

Cancers suitable for treatment as described herein include any type of solid or non-solid cancer or malignant lymphoma and especially leukaemia, sarcomas, skin cancer, bladder cancer, breast cancer, uterine cancer, ovarian cancer, prostate cancer, lung cancer, colorectal cancer, cervical cancer, liver cancer, head and neck cancer, oesophageal cancer, pancreatic cancer, renal cancer, stomach cancer and cerebral cancer. Cancers may be familial or sporadic.

In some preferred embodiments, the cancer may be breast cancer.

Anti-cancer compounds are cytotoxic compounds which inhibit the growth, division and/or proliferation of cancer cells. Anti-cancer compounds may, in some circumstances, have an effect on normal non-cancer cells in a patient. An anti-cancer compound may, for example, inhibit the cell-cycle or activate apoptosis. Suitable anti-cancer compounds which inhibit the cell cycle include DNA damaging agents and anti-mitotic agents, including inhibitors of mitotic spindle assembly.

A DNA damaging agent is a chemotherapeutic compound which induces DNA DSBs in cellular DNA, thereby inhibiting or abolishing DNA replication. Many suitable compounds are known in the art for use in the treatment of cancer, including, for example, bleomycin hydorxyurea, mitomycin and actinomycin and inhibitors of topoisomerase I and II activity, including anthracylines such as daunorubicin, doxorubicin, epirubicin, idarubicin, mitoxantrone and valrubicin, etoposide and teniposide, and members of the tecan family e.g. irinotecan, topotecan, rubitecan. DNA damaging agents may be used as described herein in any convenient form or formulation. For example, any suitable isomer, salt, solvate, chemically protected form, or prodrug of a particular DNA damaging agent may be employed.

In some preferred embodiments, the DNA damaging agent is doxorubicin ((8S, 10S)-10-(4-amino-5-hydroxy-6-methyl-tetrahydro-2H-pyran-2-yloxy)-6,8,11-trihydroxy-8-(2-hydroxyacetyl)-1-methoxy-7,8,9,10-tetrahydrotetracene-5,12-dione). Doxorubicin is a anthracycline intercalating agent which is widely used in cancer treatment under trade names such as Adriamycin™, and Rubex™.

Anti-cancer compounds which inhibit mitotic spindle assembly may, for example, bind microtubules and alter microtubule polymerization or stability leading to the inhibition cell cycle progression and eventually to apoptosis. Examples of mitotic spindle assembly inhibitors include taxanes, for example paclitaxel (taxol™: β-(benzoylamino)-α-hydroxy-,6,12b-bis(acetyloxy)-12-(benzoyloxy)-2a,3,4,4a,5,6,9,10,11,12,12a,12b-dodecahydro-4,11-dihydroxy-4a,8,13,13-tetramethyl-5-oxo-7,11-methano-1H-cyclodeca(3,4)benz(1,2-b)oxet-9-ylester,(2aR-(2a-α,4-β,4a-β,6-β,9-α(α-R*,β-S*), 11-α,12-α,12a-α,2b-α))-enzenepropanoic acid) and analogues or derivatives thereof.

Taxanes are complex esters consisting of a 15-member taxane ring system linked to a four-member oxetan ring. Preferred taxanes are those having the constituents known in the art to be required for enhancement of microtubule formation, e.g., paclitaxel and docetaxel. The structures of paclitaxel and docetaxel differ in substitutions at the C-10 taxane ring position and on the ester side chain attached at C-13. Docetaxel has t-butoxycarbonyl instead of benzoyl on the amino group of (2R,3S)-phenylisoserine moiety at the C-13 position and a hydroxyl group instead of acetoxy group at C-10. The structures of paclitaxel and docetaxel are well known in the art.

Other taxanes suitable for use as described herein are paclitaxel derivatives having structural variations along the portion of the paclitaxel molecule comprising carbons 6-12, with oxygen functions at C-7, C-9 and C-10. Many such derivatives are known in the art, and it is known that such derivatives exhibit biological activity that is comparable to the bioactivity of paclitaxel. For example, acylation of the C-7 hydroxyl group, or its replacement with hydrogen, does not significantly reduce the activity of paclitaxel. Additionally, replacement of the 10-acetoxy group with hydrogen causes only a small reduction in activity.

Reduction of the C-9 carbonyl group to an a-OH group is known to cause a slight increase in tubulin-assembly activity. Additionally, it is known that a rearrangement product with a cyclopropane ring bridging the seven and eight-position is almost as cytotoxic as paclitaxel. It has also been reported that m-substituted benzoyl derivatives are more active than their p-substituted analogues, and are often more active than paclitaxel itself.

Another paclitaxel analog suitable for use as described herein is A-nor-paclitaxel. This analog has tubulin-assembly activity that is only three times less than that of paclitaxel. A-nor-paclitaxel and paclitaxel have very similar molecular shapes, which may explain their similar tubulin-assembly activities.

Other suitable taxanes are taxasm, 7-epipaclitaxel, t-acetyl paclitaxel, 10-desacetyl-paclitaxel, 10-desacetyl-7-epipaclitaxel, 7-xylosylpaclitaxel, 10-desacetyl-7-glutarylpaclitaxel, 7-N,N-dimethylglycylpaclitaxel, 7-L-alanylpaclitaxel, and mixtures thereof.

In some preferred embodiments, the anti-cancer compound is paclitaxel.

An antibody-IL2 conjugate for use as described herein may comprise interleukin 2 (IL2) conjugated to an antibody which specifically binds to tenascin-C.

Interleukin-2 (IL2) is a secreted cytokine which is involved in immunoregulation and the proliferation of T and B lymphocytes. IL2 has been shown to have a cytotoxic effect on tumour cells and recombinant human IL2 (aldesleukin: Proleukin^{R}) has FDA approval for treatment of metastatic renal carcinoma and metastatic melanoma. The sequence of human IL2 is set out in SEQ ID NO: 11 and publicly available under sequence database reference NP_{—}000577.2 GI: 28178861.

In some preferred embodiments, the IL2 moiety of the antibody-IL2 conjugate comprises a sequence which has at least 90% sequence identity, at least 95% sequence identity or at least 98% sequence identity to the mature human IL2 sequence set out in SEQ ID NO: 11.

Sequence identity is commonly defined with reference to the algorithm GAP (Wisconsin GCG package, Accelerys Inc, San Diego USA). GAP uses the Needleman and Wunsch algorithm to align two complete sequences that maximizes the number of matches and minimizes the number of gaps. Generally, default parameters are used, with a gap creation penalty=12 and gap extension penalty=4. Use of GAP may be preferred but other algorithms may be used, e.g. BLAST (which uses the method of Altschul et al. (1990) J. Mol. Biol. 215: 405-410), FASTA (which uses the method of Pearson and Lipman (1988) PNAS USA 85: 2444-2448), or the Smith-Waterman algorithm (Smith and Waterman (1981) J. Mol Biol. 147: 195-197), or the TBLASTN program, of Altschul et al. (1990) supra, generally employing default parameters. In particular, the psi-Blast algorithm (Nucl. Acids Res. (1997) 25 3389-3402) may be used.

In some especially preferred embodiments, the IL2 moiety of the antibody-IL2 conjugate comprises the sequence of mature human IL2 set out in SEQ ID NO: 11.

The IL2 moiety may be fused upstream (N-terminal) or downstream (C-terminal) of the antibody or polypeptide component thereof.

The IL2 moiety may be connected or attached to the antibody moiety of the antibody-IL2 conjugate by any suitable covalent or non-covalent means. In preferred embodiments, the antibody-IL2 conjugate may be a fusion protein comprising IL2 and the anti-tenascin C antibody or a polypeptide component thereof (e.g. a heavy chain or a light chain of an antibody or multi-chain antibody fragment, such as a Fab. Thus, for example, the IL2 moiety may be fused to a VH domain or VL domain of the antibody. Typically the antibody, or component thereof, and IL2 moiety are joined via a peptide linker, e.g. a peptide of about 5-25 residues, e.g. 10-20 residues, preferably about 15 residues. Suitable examples of peptide linkers are well known in the art. In some embodiments, a linker may have an amino acid sequence as set out in SEQ ID NO: 12. Normally, the linker has an amino acid sequence comprising one or more tandem repeats of a motif. Typically the motif is a five residue sequence, and preferably at least 4 of the residues are Gly or Ser. Where four of the five residues is Gly or Ser, the other residue may be Ala. More preferably each of the five residues is Gly or Ser. Preferred motifs are GGGGS, SSSSG, GSGSA and GGSGG. Preferably, the motifs are adjacent in the sequence, with no intervening nucleotides between the repeats. The linker sequence may comprise or consist of between one and five, preferably three or four, repeats of the motif. For example, a linker with three tandem repeats may have one of the following amino acid sequences:

| | |
|---|---|
| GGGGSGGGGSGGGGS - | SEQ ID NO: 13 |
| SSSSGSSSSGSSSSG - | SEQ ID NO: 14 |
| GSGSAGSGSAGSGSA - | SEQ ID NO: 15 |
| GGSGGGGSGGGGSGG -. | SEQ ID NO: 16 |

In preferred embodiments, the antibody moiety of the antibody-IL2 conjugate specifically binds to tenascin-C large isoform. For example, the antibody may bind preferentially to tenascin-C large isoform relative to tenascin-C small isoform. Most preferably, the antibody binds to the Al domain of tenascin-C large isoform.

Preferred antibodies are tumour specific and bind preferentially to tumour tissue relative to normal tissue. Antibodies may, for example, bind to stroma and/or neo- and peri-vascular structures of tumour tissue preferentially to normal tissue.

Examples of suitable antibodies for use in antibody-IL2 conjugates are disclosed in WO2006/050834.

In some embodiments, the antibody moiety of an antibody-IL2 conjugate as described herein competes for binding to tenascin-C with an antibody comprising the 4A1-F16 VH domain of SEQ ID NO. 2 and the 4A1-F16 VL domain of SEQ ID NO. 4.

Competition between antibodies may be assayed easily in vitro, for example using ELISA and/or by tagging a specific reporter molecule to one antibody which can be detected in the presence of other untagged antibody(s), to enable identification of antibodiess which bind the same epitope or an overlapping epitope.

A suitable antibody for use in an antibody-IL2 conjugate as described herein may comprise an antibody antigen binding site comprising a VH domain and a VL domain,
the VH domain comprising a VH CDR1 of SEQ ID NO. 5, a VH CDR2 of SEQ ID NO. 6 and a VH CDR3 of SEQ ID NO. 7; and
the VL domain comprising a VL CDR1 of SEQ ID NO. 8, a VL CDR2 of SEQ ID NO. 9 and a VL CDR3 of SEQ ID NO. 10.

In some preferred embodiments, the antibody may comprise an antibody antigen binding site comprising the 4A1-F16 VH domain of SEQ ID NO. 2 and the 4A1-F16 VL domain of SEQ ID NO. 4.

Variants of these VH and VL domains and CDRs may also be employed in antibodies for use in antibody-IL2 conjugates as described herein as described herein. Suitable variants can be obtained by means of methods of sequence alteration or mutation and screening.

Particular variants for use as described herein may include one or more amino acid sequence alterations (addition, deletion, substitution and/or insertion of an amino acid residue), maybe less than about 20 alterations, less than about 15 alterations, less than about 10 alterations or less than about 5 alterations, 4, 3, 2 or 1. Alterations may be made in one or more framework regions and/or one or more CDRs. In particular, alterations may be made in VH CDR1, VH CDR2 and/or VH CDR3, especially VH CDR3.

Administration of the anti-cancer compound, antibody-IL2 conjugate and compositions comprising one or both of these molecules is preferably in a “therapeutically effective amount”, this being sufficient to show benefit to a patient. Such benefit may be at least amelioration of at least one symptom. The actual amount administered, and rate and time-course of administration, will depend on the nature and severity of what is being treated. Prescription of treatment, e.g. decisions on dosage etc, is within the responsibility of general practitioners and other medical doctors.

The precise dose will depend upon a number of factors, the size and location of the area to be treated, the precise nature of the antibody-IL2 conjugate (e.g. whole antibody, fragment or diabody). A typical antibody-IL2 conjugate dose will be in the range 0.5 mg to 100 g for systemic applications, and 10 μg to 1 mg for local applications. Typically, the antibody moiety of the conjugate will be a whole antibody, preferably the IgG1 or IgG4 isotype. This is a dose for a single treatment of an adult patient, which may be proportionally adjusted for children and infants, and also adjusted for other antibody formats in proportion to molecular weight. Appropriate doses and regimens for Anti-cancer compounds are well known in the art.

Treatments may be repeated at daily, twice-weekly, weekly or monthly intervals, at the discretion of the physician.

The antibody-IL2 conjugate and the anti-cancer compound may be administered sequentially or simultaneously in accordance with any suitable regimen.

The antibody-IL2 conjugate and the anti-cancer compound will usually be administered to an individual in the form of pharmaceutical compositions, which may comprise at least one component in addition to the active compound.

Suitable components include a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. intravenous.

The antibody-IL2 conjugate and the anti-cancer compound may be formulated in separate pharmaceutical compositions or, where appropriate, in the same pharmaceutical composition.

Another aspect of the invention provides a pharmaceutical composition for use in the treatment of cancer comprising an anti-cancer compound and an antibody-IL2 conjugate comprising interleukin 2 (IL2) conjugated to an antibody which specifically binds to tenascin-C.

Another aspect of the invention provides a method of making a pharmaceutical composition for use in the treatment of cancer comprising formulating an anti-cancer compound and an antibody-IL2 conjugate comprising interleukin 2 (IL2) conjugated to an antibody which specifically binds to tenascin-C.

Pharmaceutical compositions for oral administration may be in tablet, capsule, powder or liquid form. A tablet may comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Another aspect of the invention provides a therapeutic kit for use in the treatment of cancer comprising an anti-cancer compound and an antibody-IL2 conjugate comprising interleukin 2 (IL2) conjugated to an antibody which specifically binds to tenascin-C.

The components of a kit (i.e. the anti-cancer compound and antibody-IL2 conjugate) are sterile and in sealed vials or other containers. A kit may further comprise instructions for use of the components in a method described herein. The components of the kit may be comprised or packaged in a container, for example a bag, box, jar, tin or blister pack.

### Terminology

Antibody

This describes an immunoglobulin whether natural or partly or wholly synthetically produced. The term also covers any polypeptide or protein having a binding domain which is, or is substantially homologous to, an antibody binding domain. Examples of antibodies are the immunoglobulin isotypes and their isotypic subclasses; fragments which comprise an antigen binding domain such as Fab, scFv, Fv, dAb, Fd; and diabodies.

It is possible to take monoclonal and other antibodies and use techniques of recombinant DNA technology to produce other antibodies or chimeric molecules which retain the specificity of the original antibody. Such techniques may involve introducing DNA encoding the immunoglobulin variable region, or the complementarity determining regions (CDRs), of an antibody to the constant regions, or constant regions plus framework regions, of a different immunoglobulin. See, for instance, EP-A-184187, GB 2188638A or EP-A-239400. A hybridoma or other cell producing an antibody may be subject to genetic mutation or other changes, which may or may not alter the binding specificity of antibodies produced.

As antibodies can be modified in a number of ways, the term “antibody” should be construed as covering any specific binding member or substance having a binding domain with the required specificity. Thus, this term covers antibody fragments, derivatives, functional equivalents and homologues of antibodies, including any polypeptide comprising an immunoglobulin binding domain, whether natural or wholly or partially synthetic. Chimeric molecules comprising an immunoglobulin binding domain, or equivalent, fused to another polypeptide are therefore included. Cloning and expression of chimeric antibodies are described in EP-A-0120694 and EP-A-0125023.

It has been shown that fragments of a whole antibody can perform the function of binding antigens. Examples of binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment (Ward, E. S. et al., Nature 341, 544-546 (1989)) which consists of a VH domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al, Science, 242, 423-426, 1988; Huston et al, PNAS USA, 85, 5879-5883, 1988); (viii) bispecific single chain Fv dimers (PCT/US92/09965) and (ix) “diabodies”, multivalent or multispecific fragments constructed by gene fusion (WO94/13804; P. Holliger et al, Proc. Natl. Acad. Sci. USA 90 6444-6448, 1993). Fv, scFv or diabody molecules may be stabilised by the incorporation of disulphide bridges linking the VH and VL domains (Y. Reiter et al. Nature Biotech 14 1239-1245 1996). Minibodies comprising an scFv joined to a CH3 domain may also be made (S. Hu et al, Cancer Res. 56 3055-3061 1996).

Diabodies are multimers of polypeptides, each polypeptide comprising a first domain comprising a binding region of an immunoglobulin light chain and a second domain comprising a binding region of an immunoglobulin heavy chain, the two domains being linked (e.g. by a peptide linker) but unable to associate with each other to form an antigen binding site: antigen binding sites are formed by the association of the first domain of one polypeptide within the multimer with the second domain of another polypeptide within the multimer (WO94/13804).

Antigen Binding Domain

This describes the part of an antibody which comprises the area which specifically binds to and is complementary to part or all of an antigen. Where an antigen is large, an antibody may only bind to a particular part of the antigen, which part is termed an epitope. An antigen binding domain may be provided by one or more antibody variable domains (e.g. a so-called Fd antibody fragment consisting of a VH domain). Preferably, an antigen binding domain comprises an antibody light chain variable region (VL) and an antibody heavy chain variable region (VH).

Specific

This may be used to refer to the situation in which one member of a specific binding pair will not show any significant binding to molecules other than its specific binding partner(s). For example, an antibody specific for Tenascin-C may show little or no binding to other components of the extracellular matrix such as fibronectin. Similarly, an antibody specific for Tenascin-C large isoform may show little or no binding to Tenascin-C small isoform. The term is also applicable where e.g. an antigen binding domain is specific for a particular epitope which is carried by a number of antigens, in which case the specific binding member carrying the antigen binding domain will be able to bind to the various antigens carrying the epitope.

Comprise

This is generally used in the sense of include, that is to say permitting the presence of one or more features or components.

By “substantially as set out” it is meant that the relevant CDR or

VH or VL domain of the invention will be either identical or highly similar to the specified regions of which the sequence is set out herein. By “highly similar” it is contemplated that from 1 to 5, preferably from 1 to 4 such as 1 to 3 or 1 or 2, or 3 or 4, substitutions may be made in the CDR and/or VH or VL domain.

The structure for carrying a CDR of the invention will generally be of an antibody heavy or light chain sequence or substantial portion thereof in which the CDR is located at a location corresponding to the CDR of naturally occurring VH and VL antibody variable domains encoded by rearranged immunoglobulin genes. The structures and locations of immunoglobulin variable domains and CDRs may be determined by reference to (Kabat, E. A. et al, Sequences of Proteins of Immunological Interest. 4th Edition. US Department of Health and Human Services. 1987, and updates thereof, now available on the Internet (http://immuno.bme.nwu.edu)).

Various further aspects and embodiments of the present invention will be apparent to those skilled in the art in view of the present disclosure. All documents and database entries mentioned in this specification are incorporated herein by reference in their entirety.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

Certain aspects and embodiments of the invention will now be illustrated by way of example and with reference to the figures described above and tables described below.

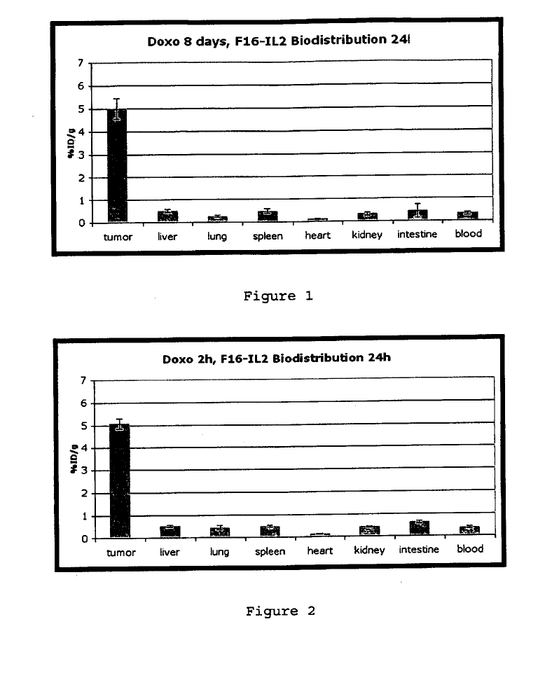

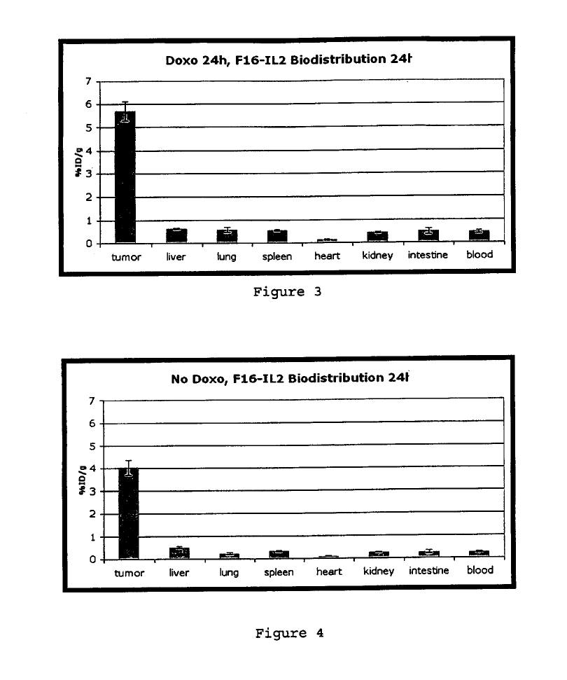

FIGS. 1 to 4 show the biodistribution of labelled F16-IL2 administered after pre-injection with doxorubicin.

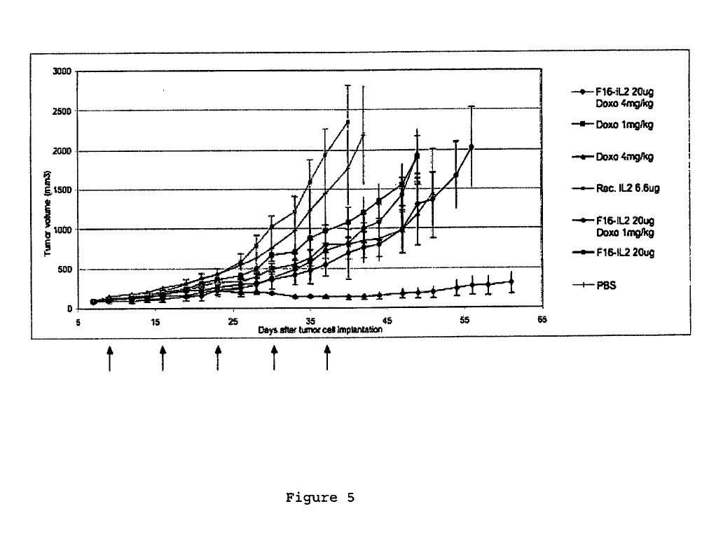

FIG. 5 shows the effect of doxorubicin, F16-IL2 and recombinant IL2 on the MDA-MB231 human breast cancer tumors implanted in nude mice.

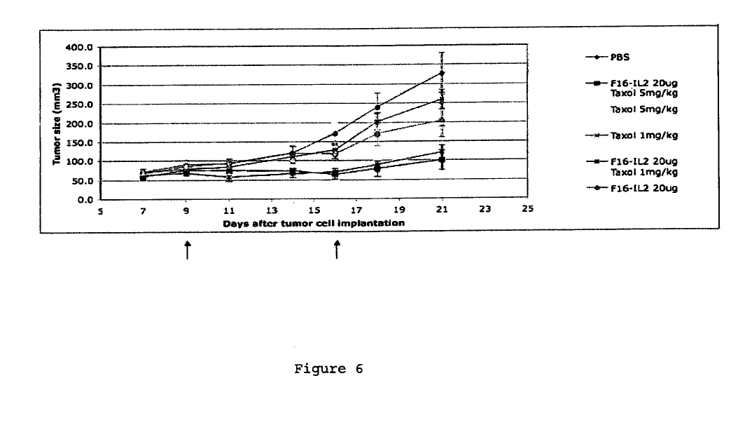

FIG. 6 shows the effect of paclitaxel (taxol™) and F16-IL2 on the MDA-MB231 human breast cancer tumors implanted in nude mice.

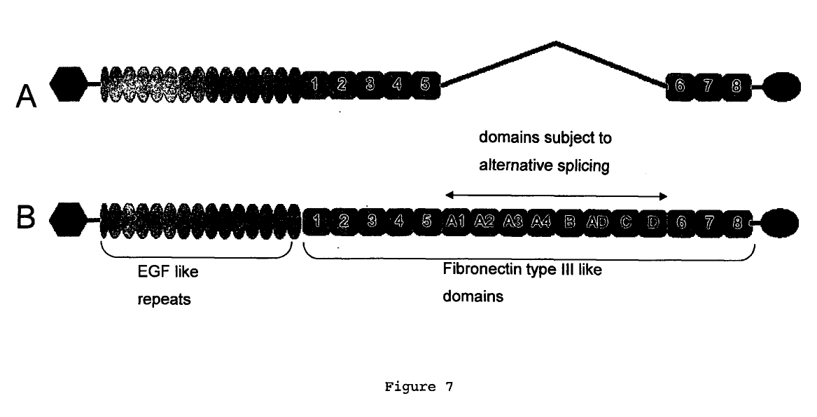

FIG. 7 shows a schematic representation of the small (A) and large (B) tenascin-C isoform. Several fibronectin type III like domains are subject to alternative splicing, either being included (B) or omitted (A) in the molecule. The amino acid sequence and encoding nucleotide sequence of tenascin C are publically available under sequence database references NP_{—}002151.1 GI:4504549 and NM_{—}002160.1 GI:4504548, respectively.

### EXPERIMENTS

Biodistribution

The in vivo targeting performance was evaluated by biodistribution analysis. Tumor-bearing mice were obtained by injecting 10̂7 MDA-MB-231 human breast cancer cells s.c. in 10- to 12-week old Balb/c nude female mice (Charles River Laboratories). Mice were grouped (n≧5) when tumors were clearly palpable and injected i.v. in the lateral tail vein with 10 mg/kg doxorubicin 8 days, 24 h or 2 h prior to biodistribution. The control group was exempted from doxorubicin administration. Purified F16-IL2 was radioiodinated and injected into the lateral tail vein of all mice. Mice were sacrificed 24 h after injection (12.5 μg, 3.3 μCi per mouse). Organs were weighed and radioactivity was counted with a Packard Cobra gamma counter. Radioactivity content of representative organs was expressed as the percentage of the injected dose per gram of tissue (%ID/g).

The results of these experiments showed that pre-injection of doxorubicin does not impair the tumor targeting of the immunocytokine (FIGS. 1 to 4).

Therapy

Tumor-bearing mice were obtained by injecting 2*10̂7 MDA-MB-231 human breast cancer cells s.c. in 10- to 12-week old Balb/c nude female mice (Charles River Laboratories). Mice were grouped (n=5) 9 days after tumor cell implantation when tumors were clearly palpable and injected i.v. in the lateral tail vein with saline, 20 μg F16-IL2 (corresponding to 6.6 μg IL2), 6.6 μg recombinant IL2 (Proleukin®, and 4 mg/kg or 1 mg/kg Doxorubicin in a maximum volume of 250 μl. Mice were monitored daily and tumor growth was measured three times weekly with a caliper using the following formula: volume=length×width2×0.5. Animals were sacrificed when tumors reached a volume >2000 mm3 or when tumors became necrotic according to Swiss regulations and under a project license granted by the

Veterinäramt des Kantons Zürich (198/2005). Tumor sizes are expressed as mean±SE.

A synergistic effect was observed between F16-IL2 and doxorubicin in the in MDA-MB231 human breast cancer model implanted in nude mice (FIG. 5). It was also observed that higher doses of doxorubicin were even more effective than low doses.

Tumor-bearing mice produced in the same way were injected i.v. in the lateral tail vein with saline, 20 μg F16-IL2 (corresponding to 6.6 μg IL2), and 1 mg/kg or 5 mg/kg taxol™ in a maximum volume of 250 μl. Mice were monitored and tumor growth measured as described above.

A synergistic effect was observed between F16-IL2 and taxol™ in the in MDA-MB231 human breast cancer model implanted in nude mice (FIG. 6).

Sequences
SEQ ID NO: 1.
4A1-F16 VH domain nucleotide sequence
GAG GTG CAG CTG TTG GAG TCT GGG GGA GGC TTG GTA CAG CCT GGG GGG TCC
CTG AGA CTC TCC TGT GCA GCC TCT GGA TTC ACC TTT AGC CGG TAT GGT GCG
AGC TGG GTC CGC CAG GCT CCA GGG AAG GGG CTG GAG TGG GTC TCA GCT ATT
AGT GGT AGT GGT GGT AGC ACA TAC TAC GCA GAC TCC GTG AAG GGC CGG TTC
ACC ATC TCC AGA GAC AAT TCC AAG AAC ACG CTG TAT CTG CAA ATG AAC AGC
CTG AGA GCC GAG GAC ACG GCC GTA TAT TAC TGT GCG AAA GCG CAT AAT GCT
TTT GAC TAC TGG GGC CAG GGA ACC CTG GTC ACC GTG TCG AGA
SEQ ID NO: 2
4A1-F16 VH domain amino acid sequence
EVQLLESGGG LVQPGGSLRL SCAASGFTFS RYGASWVRQA PGKGLEWVSA ISGSGGSTYY
ADSVKGRFTI SRDNSKNTLY LQMNSLRAED TAVYYCAKAH NAFDYWGQGT
LVTVSREVQLLESGGG LVQPGGSLRL SCAASGFTFS RYGASWVRQA PGKGLEWVSA
ISGSGGSTYY ADSVKGRFTI SRDNSKNTLY LQMNSLRAED TAVYYCAKAH NAFDYWGQGT
LVTVSR
SEQ ID NO: 3
4A1-F16 VL domain nucleotide sequence
TCG TCT GAG CTG ACT CAG GAC CCT GCT GTG TCT GTG GCC TTG GGA CAG ACA
GTC AGG ATC ACA TGC CAA GGA GAC AGC CTC AGA AGC TAT TAT GCA AGC TGG
TAC CAG CAG AAG CCA GGA CAG GCC CCT GTA CTT GTC ATC TAT GGT AAA AAC
AAC CGG CCC TCA GGG ATC CCA GAC CGA TTC TCT GGC TCC AGC TCA GGA AAC
ACA GCT TCC TTG ACC ATC ACT GGG GCT CAG GCG GAA GAT GAG GCT GAC TAT
TAC TGT AAC TCC TCT GTT TAT ACT ATG CCG CCC GTG GTA TTC GGC GGA GGG
ACC AAG CTG ACC GTC CTA GGC
SEQ ID NO: 4
4A1-F16 VL domain amino acid sequence
SSELTQDPAV SVALGQTVRI TCQGDSLRSY YASWYQQKPG QAPVLVIYGK NNRPSGIPDR
FSGSSSGNTA SLTITGAQAE DEADYYCNSS VYTMPPVVFG GGTKLTVLG
SEQ ID NO: 5
4A1-F16 VH CDR1 amino acid sequence
RYGAS
SEQ ID NO: 6
4A1-F16 VH CDR2 amino acid sequence
AISGSGGSTYYADSVKG
SEQ ID NO: 7
4A1-F16 VH CDR3 amino acid sequence
AHNAFDY
SEQ ID NO: 8
4A1-F16 VL CDR1 amino acid sequence
QGDSLRSYYAS
SEQ ID NO: 9
4A1-F16 VL CDR2 amino acid sequence
GKNNRPS
SEQ ID NO: 10
4A1-F16 VL CDR3 amino acid sequence
NSSVYTMPPVV
SEQ ID NO: 11
hIL2 precursor sequence (mature hIL2: residues 7-150)
MYRMQLLSCI ALSLALVTNS APTSSSTKKT QLQLEHLLLD LQMILNGINN YKNPKLTRML
TFKFYMPKKA TELKHLQCLE EELKPLEEVL NLAQSKNFHL RPRDLISNIN VIVLELKGSE
TTFMCEYADE TATIVEFLNR WITFCQSIIS TLT
SEQ ID NO: 12
Peptide linker amino acid sequence
GGGGSGGGGSGGGG
SEQ ID NO: 13
Peptide linker amino acid sequence
GGGGSGGGGSGGGGS
SEQ ID NO: 14
Peptide linker amino acid sequence
SSSSGSSSSGSSSSG
SEQ ID NO: 15
Peptide linker amino acid sequence
GSGSAGSGSAGSGSA
SEQ ID NO: 16
Peptide linker amino acid sequence
GGSGGGGSGGGGSGG

1.-24. (canceled)

25. A method of treating cancer comprising:
administering docetaxel and an antibody-interleukin 2 (IL2) conjugate to an individual in need thereof,
wherein the antibody-IL2 conjugate comprises IL2 conjugated to an antibody which specifically binds to tenascin-C.

26. A method according to claim 25 wherein the antibody specifically binds to the tenascin-C large isoform.

27. A method according to claim 26 wherein the antibody specifically binds to the Al domain of tenascin-C large isoform.

28. A method according to claim 27 wherein the antibody competes for binding to tenascin-C large isoform with an antibody comprising the 4A1-F16 VH domain of SEQ ID NO: 2 and the 4A1-F16 VL domain of SEQ ID NO: 4.

29. A method according to claim 28 wherein the antibody comprises an antibody antigen binding site comprising a VH domain and a VL domain,
the VH domain comprising a VH CDR1 of SEQ ID NO: 5, a VH CDR2 of SEQ ID NO: 6 and a VH CDR3 of SEQ ID NO: 7; and
the VL domain comprising a VL CDR1 of SEQ ID NO: 8, a VL CDR2 of SEQ ID NO: 9 and a VL CDR3 of SEQ ID NO: 10.

30. A method according to claim 29 wherein the antibody comprises an antibody antigen binding site comprising the 4A1-F16 VH domain of SEQ ID NO: 2 and the 4A1-F16 VL domain of SEQ ID NO: 4.

31. A method of treating a tenascin C-expressing cancer comprising: sequentially administering docetaxel or an analogue or derivative thereof, and an antibody-interleukin 2 (IL2) conjugate to an individual in need thereof, said docetaxel or said analogue or derivative being administered before said conjugate, wherein the antibody-IL2 conjugate comprises IL2 conjugated to an antibody which specifically binds to tenascin-C present in said cancer cells, and wherein the antibody competes for binding to tenascin-C large isoform with an antibody comprising the 4A1-F16 VH domain of SEQ ID NO:2 and the 4A1-F16 VL domain of SEQ ID NO:4 and wherein said docetaxel and said conjugate act synergistically to kill cancer cells.

32. The method according to claim 31 wherein the antibody comprises an antibody antigen binding site comprising a VH domain and a VL domain, the VH domain comprising a VH CDR1 of SEQ ID NO:5, a VH CDR2 of SEQ ID NO:6 and a VH CDR3 of SEQ ID NO:7; and the VL domain comprising a VL CDR1 of SEQ ID NO:8, a VL CDR2 of SEQ ID NO:9 and a VL CDR3 of SEQ ID NO:10.

33. A method of treating a tenascin C-expressing cancer comprising: sequentially administering docetaxel or an analogue or derivative thereof, and an antibody-interleukin 2 (IL2) conjugate to an individual in need thereof said docetaxel or said analogue or said derivative being administered before said conjugate, wherein the antibody-IL2 conjugate comprises IL2 conjugated to an antibody which specifically binds to tenascin-C present in said cancer cells and, wherein the antibody comprises an antibody antigen binding site comprising the 4A1-F16 VH domain of SEQ ID NO:2 and the 4A1-F16 VL domain of SEQ ID NO:4, and wherein said docetaxel and said conjugate act synergistically to kill cancer cells.

34. The method according to claim 31 wherein the cancer is breast cancer.

35. The method according to claim 31 wherein the cancer is lung cancer.

36. A kit for use in a treatment of cancer comprising docetaxel and an antibody-IL2 conjugate comprising interleukin 2 (IL2) conjugated to an antibody selected from the group consisting of (i) antibody that competes for binding to tenascin-C large isoform with an antibody comprising the 4A1-F16 VH domain of SEQ ID NO:2 and the 4A1-F16 VL domain of SEQ ID NO:4 and (ii) antibody comprising an antigen binding site comprising the 4A1-F16 VH domain of SEQ ID NO:2 and the 4A1-F16 VL domain of SEQ ID NO:4, which specifically binds to cancer cells expressing tenascin-C.


SEQUENCE LISTING (20 sequences)

SEQ ID NO: 1 - DNA, 348 bp, Homo sapiens
gaggtgcagctgttggagtctgggggaggcttggtacagcctggggggtccctgagactctcctgtgcagcctctggattcacctttagccggtatggtgcgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagcgcataatgcttttgactactggggccagggaaccctggtcaccgtgtcgaga

SEQ ID NO: 2 - protein, 116 aa, Homo sapien
EVQLLESGGGLVQPGGSLRLSCAASGFTFSRYGASWVRQAPGKGLEWVSAISGSGGSTYYADSVKGRFTISRDNSKNTLYLQMNSLRAEDTAVYYCAKAHNAFDYWGQGTLVTVSR

SEQ ID NO: 3 - DNA, 327 bp, Homo Sapien
tcgtctgagctgactcaggaccctgctgtgtctgtggccttgggacagacagtcaggatcacatgccaaggagacagcctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtaactcctctgtttatactatgccgcccgtggtattcggcggagggaccaagctgaccgtcctaggc

SEQ ID NO: 4 - protein, 109 aa, Homo sapien
SSELTQDPAVSVALGQTVRITCQGDSLRSYYASWYQQKPGQAPVLVIYGKNNRPSGIPDRFSGSSSGNTASLTITGAQAEDEADYYCNSSVYTMPPVVFGGGTKLTVLG

SEQ ID NO: 5 - protein, 5 aa, Homo sapien
RYGAS

SEQ ID NO: 6 - protein, 17 aa, Homo sapien
AISGSGGSTYYADSVKG

SEQ ID NO: 7 - protein, 7 aa, Homo sapien
AHNAFDY

SEQ ID NO: 8 - protein, 11 aa, Homo sapien
QGDSLRSYYAS

SEQ ID NO: 9 - protein, 7 aa, Homo sapien
GKNNRPS

SEQ ID NO: 10 - protein, 11 aa, Homo sapien
NSSVYTMPPVV

SEQ ID NO: 11 - protein, 153 aa, Homo sapien
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 12 - protein, 14 aa, Artificial Sequence
  Synthetic sequence
GGGGSGGGGSGGGG

SEQ ID NO: 13 - protein, 15 aa, Artificial Sequence
  Synthetic sequence
GGGGSGGGGSGGGGS

SEQ ID NO: 14 - protein, 15 aa, Artificial Sequence
  Synthetic sequence
SSSSGSSSSGSSSSG

SEQ ID NO: 15 - protein, 15 aa, Artificial Sequence
  Synthetic sequence
GSGSAGSGSAGSGSA

SEQ ID NO: 16 - protein, 15 aa, Artificial Sequence
  Synthetic sequence
GGSGGGGSGGGGSGG

SEQ ID NO: 17 - protein, 5 aa, Artificial Sequence
  Synthetic sequence
GGGGS

SEQ ID NO: 18 - protein, 5 aa, Artificial Sequence
  Synthetic sequence
SSSSG

SEQ ID NO: 19 - protein, 5 aa, Artificial Sequence
  Synthetic sequence
GSGSA

SEQ ID NO: 20 - protein, 5 aa, Artificial Sequence
  Synthetic sequence
GGSGG